(19) United States
(12) Patent Application Publication
(10) Pub. No.: US 2016/0289662 A1
(43) Pub. Date: Oct. 6, 2016

(54) COMPOSITION FOR DISPERSING BIOLOGICAL TISSUE

(71) Applicant: KURASHIKI BOSEKI KABUSHIKI KAISHA, Kurashiki-shi Okayama (JP)

(72) Inventors: Isao MIYAGAWA, Kyotanabe-shi, Kyoto (JP); Seiko KAWAMURA, Shijonawate-shi, Osaka (JP); Hisayuki KOBAYASHI, Osaka-shi, Osaka (JP)

(73) Assignee: KURASHIKI BOSEKI KABUSHIKI KAISHA, Kurashiki-shi, Okayama (JP)

(21) Appl. No.: 15/035,614

(22) PCT Filed: Oct. 14, 2014
(86) PCT No.: PCT/JP2014/077356

§ 371 Date: May 10, 2016

(30) Foreign Application Priority Data
Nov. 12, 2013 (JP) 2013-234036

(51) Int. Cl. C12N 9/64; C12N 5/09; C12N 9/76

(52) U.S. Cl. CPC C12N 9/6491; C12N 9/6427; C12Y 304/24007; C12Y 304/21004; C12N 5/0693; C12N 2509/00; C12N 2503/02

(57) ABSTRACT

The purpose of the present invention is to acquire a highly proliferative cell at a high efficiency from a sample derived from a biological tissue. Provided is a composition for dispersing a biological tissue, wherein a solution formulation of the composition has a collagenase activity of 0.30-10 U/mL, said collagenase activity being determined by a method for measuring FALGPA-decomposing activity, and a trypsin activity of 0-30 U/mL at a formulation concentration of the composition, said trypsin activity being determined by a method for measuring BASE hydrolytic activity.

### TECHNICAL FIELD

The present invention relates to a composition for dispersing biological tissue. The present invention also relates to a method for evaluating a result of culturing cell which is embedded in a droplet gel. In addition, the present invention relates to a method for obtaining a cell from biological tissue. The present invention also relates to a kit for carrying out the above method.

### BACKGROUND ART

It is well known that an effect of anticancer agent varies markedly among patients, and that response rates of anticancer agents are less than 50% with the exception of some agents, while anticancer agents may show strong side effect by acting on normal cell. Therefore, it is required that an effect of anticancer agent to be administered to a patient is assessed before the administration of the anticancer agent to the patient to reduce physical and economic load of the patient, and to avoid the loss of therapeutic opportunities.

As an evaluation method of such an anticancer effect, a method including proliferating a cancer cell in three dimensions in a droplet gel mimicking biological body, and then contacting an anticancer agent to evaluate a result of proliferation (Patent Documents 1 to 11), is known. In the method, it is required to obtain a cell which proliferates in the droplet gel as same as in vivo.

Patent Documents 1 to 7 describe that an enzyme, such as collagenase, hyaluronidase, deoxyribonuclease, elastase, and dispase, is used in a process of obtaining a cell from a sample derived from biological tissue. In addition, Patent Documents 9 to 11 describe that biological tissue is treated with mixed enzymes including one or more proteases selected from the group consisting of clostridial neutral protease, thermolysin, and dispase; and one or more collagenases selected from the group consisting of collagenase I, collagenase II, and collagenase IV.

Furthermore, Non-Patent Documents 1 to 21 describe an enzyme of dispersing biological cells. These documents describe that a sample from biological tissue is treated with an enzyme such as collagenase type I, collagenase type II, collagenase type III, collagenase type IV, trypsin, hyaluronidase, and neuraminidase.

### PRIOR ART DOCUMENTS

### Patent Documents

Patent Document 1: JP 2879978 B
Patent Document 2: JP H03-285696 A
Patent Document 3: JP H07-31470 A
Patent Document 4: JP H07-241190 A.
Patent Document 5: JP H10-115612 A
Patent Document 6: JP 2003-9853 A
Patent Document 7: JP 2008-11797 A
Patent Document 8: JP 2005-95058 A
Patent Document 9: JP 2010-227088 A
Patent Document 10: JP 2011-115106 A
Patent Document 11: WO 2011/090068 A1

### Non-Patent Documents

Non-Patent Document 1: Zhou, J, Belay, L., Solomon, M., Chan, C., Clarke, S. and Christopherson, R.: Colorectal
Cancer Cell Surface Protein Profiling Using an Antibody Microarray and Fluorescence Multiplexing., J Vis Exp 55, e3322, 2011
Non-Patent Document 2: Quintana, B., Shackleton, M., Foster, H., Fullen, D., Sabel, M., Johnson, T. and Morrison, S.: Phenotypic Heterogeneity Among Tumorigenic Melanoma Cells from Patients that is Reversible and Not Hierarchically Organized., Cancer Cell Vol. 18, 510, 2010
Non-Patent Document 3: Kim, M., Evans, D., Wang, H., Abbruzzese, J., Fleming, J. and Gallick, G.: Generation of Orthotopic and Heterotopic Human Pancreatic Cancer Xenografts in Immunodeficient Mice., Nat Protoc 4, 1670, 2009
Non-Patent Document 4: Sauvageot, C., Weatherbee, J., Kesari, S., Winters, S., Barnes, J., Dellagatta, J., Ramakrishna, N., Stiles, C., Kung, A., Kieran, M. and Wen, P.: Efficacy of the HSP90 Inhibitor 17-AAG in Human Glioma Cell Lines and Tumorigenic Glioma Stem Cells., Neuro Oncol Vol. 11, 109, 2009
Non-Patent Document 5: Liu, R., Wang, X., Chen, G., Dalerba, P., Gurney, A., Hoey, T., Sherlock, G., Lewicki, J., Shedden, K. and Clarke, M.: The Prognostic Role of a Gene Signature from Tumorigenic Breast-Cancer Cells., N Engl j Med 356, 217, 2007
Non-Patent Document 6: Nakashiro Koh-Ichi, Hara Shingo, Shinohara Yuji, Oyasu Miho, Kawamata Hitoshi, Shintani Satoru, Hamakawa Hiroyuki, Oyasu Ryoichi: Phenotypic switch from paracrine to autocrine role of hepatocyte growth factor in an androgen-independent human prostatic carcinoma cell line, CWR22R, Am J Pathol 165, 533-40, 2004
Non-Patent Document 7: Nishio Jun, Iwasaki Hiroshi, Ishiguro Masko, Ohjimi Yuko, Fujita Chikako, Isayama Teruto, Naito Masatoshi, Oda Yoshinao, Kaneko Yasuhiko, Kikuchi Masahiro: Establishment of a new human synovial sarcoma cell line, FU-SY-1, that expresses c-Met receptor and its ligand hepatocyte growth factor, Int J Oncol 21, 17-23, 2002
Non-Patent Document 8: Emenaker N, Calaf G, Cox D, Basson M and Qureshi N: Short chain fatty acids differentially modulate cellular phenotype and c-myc protein levels in primary human nonmalignant and malignant colonocytes, J Nutr 46, 96-105, 2001
Non-Patent Document 9: MacLeod, R: Rapid Monolayer Primary Cell Culture from Tissue Biopsy, Cell & Tissue Culture: Laboratory Procedures Vol. 1, Doyle, A., Griffiths, J., and Newell, D., John Wiley and Sons Ltd, 3E:2.1, 1995
Non-Patent Document 10: Hague, A and Paraskeva, C: Colon Adenocarcinoma Cells, Cell & Tissue Culture.: Laboratory Procedures Vol. 1, Doyle, A., Griffiths, J., and Newell, D., John Wiley and Sons, Ltd., 12C:1.1, 1995
Non-Patent Document 11: Beaunain, R., Mainquene, C., Brouty-Boye, D., Planchon, P., and Magniew, V.: “Normal” Breast Cells Adjacent to a Tumor Grown in Long-term Three Dimensional Culture, In Vitro Cell Dev Biol 29, 100, 1993
Non-Patent Document 12: Kruse, C., Mitchell, D., Kleinschmidt-DeMasteis, B, Franklin, W., Morse, H., Spector, E., and Lillehei, K.: Characterization of a Continuous Human Glioma Cell Line DBTRG-CSMG: Growth Kinetics, Karyotype, Receptor Expression and Tumor Suppressor Gene Analyses, In Vitro Cell Dev Biol 28, 609, 1992
Non-Patent Document 13: Emerman, J. and Wilkinson, D. Routine Culturing of Normal, Dysplastic and Malignant Human. Mammary Epithelial Cells from Small Tissue Samples, In Vitro Cell Dev Biol 26, 1186, 1990
Non-Patent Document 14: Boyd, J., Rinehart Jr., C., Walton, L., Siegal, G. and Kaufman, D.: Ultrastructural Characterization of Two New Human Endometrial Carcinoma Cell Lines and Normal Human Endometrial Epithelial Cells Cultured on Extracellular Matrix, In Vitro Cell Dev Biol 26, 701, 1990
Non-Patent Document 15: Sheela S, Riccardi V M, Ratner N: Angiogenic and invasive properties of neurofibroma Schwann cells, J Cell Biol 111, 645-53, 1990
Non-Patent Document 16: Sacks, P., Parnes, S., Gallick, G., Mansouri, Z., Lichtner, R., Satya-Prakash, K., Pathak, S, and Parsons, D.: Establishment and Characterization of Two New Squamous Cell Carcinoma Cell Lines Derived from Tumors of the Head and Neck, Cancer Res 48, 2858, 1988
Non-Patent Document 17: Brattain, M., Marks, M., McCombs, J., Finely, W., and Brattain, D.: Characterization of Human Colon Carcinoma Cell Lines Isolated From a Single Primary Tumour, Er J Cancer 47, 373, 1983
Non-Patent Document 18: Friedman, E., Higgins, P., Lipkin, M., Shinya, H., and Gelb, A.: Tissue Culture of Human Epithelial Cells from Benign. Colonic Tumors, In Vitro 17, 632, 1981
Non-Patent Document 19: Leung, C., and Shiu, R. Morphological and Proliferative Characteristics of Human Breast Tumor Cells Cultured on Plastic and in Collagen Matrix, In Vitro 18, 476, 1981
Non-Patent Document 20: Creasey, A., Smith, H., Hackett, A., Fukuyama, K., Epstein, W., and Madin, S.: Biological Properties of Human Melanoma Cells in Culture, In Vitro 15, 342, 1979
Non-Patent Document 21: Lasfargues, E.: Tissue Culture Methods/Applications, Kruse, P., and Patterson, M., Academic Press, 45, 1973

### SUMMARY OF THE INVENTION

### Problems to be Solved by the Invention

However, a cell prepared from a sample derived from biological tissue by a conventional method has not often proliferated as expected. In addition, high efficiency of obtaining the cell is required because only a small amount of sample derived from biological tissue may be obtained. Accordingly, an object of the present invention is to obtain a cell having high proliferation property from a sample derived from biological tissue with high efficiency.

### Means for Solving the Problems

The present inventors have intensively studied. As a result, they have found that trypsin, which is included in a composition used for dispersing a sample from biological tissue, and is thought to contribute to a solubilization of the tissue, inhibits collagenase and other useful enzymes. In addition, they have also found that high trypsin activity shows high cytotoxicity and decreases proliferation property of the obtained cell. The present inventors have further intensively studied based on the above findings. As a result, they have found that a cell having high proliferation property is obtained with high efficiency by dispersing a sample derived from biological tissue with a composition, which is different from a conventional composition, having reduced trypsin activity and high collagenase activity. A cell dispersion has been conventionally carried out by using a composition having high trypsin activity because an efficiency of obtaining cell tends to be low when dispersion of sample from biological tissues is insufficient. Contrary to the conventional manner, the present invention is a breakthrough having found that an efficiency of obtaining cell having high proliferation property is increased by reducing trypsin activity.

That is, in the first aspect, the present invention provides a composition for dispersing biological tissue, where a collagenase activity of the composition in a formulation solution is 0.30 U/mL to 10 U/mL as determined by a method for measuring FALGPA-degrading activity, and where a trypsin activity of the composition in the formulation solution is 0 U/mL to 30 U/mL as determined by a method for measuring BAEE hydrolytic activity.

In the second aspect, the present invention provides the composition of the first aspect, for a drug assessment.

In the third aspect, the present invention provides the composition of the first or second aspect, where the biological tissue is cancer tissue.

In the fourth aspect, the present invention provides a method for obtaining a cell derived from biological tissue, including treating a sample derived from the biological tissue with the composition of any one of the first to third aspects.

In the fifth aspect, the present invention provides a method for evaluating a cell culture result, where the cell is treated with the composition of any one of the first to third aspects.

In the sixth aspect, the present invention provides the method of the fifth aspect, where the cell culture result is a result from two-dimensional culture.

In the seventh aspect, the present invention provides the method of the fifth aspect, where the cell culture result is a result from three-dimensional culture.

In the eighth aspect, the present invention provides the method of the seventh aspect, where the three-dimensional culture is carried out in a droplet gel.

In the ninth aspect, the present invention provides a kit for carrying out the method of the fourth or fifth aspect, including the composition of any one of the first to third aspects.

### Effects of the Invention

A cell having a less amount of tissue, which is adhered around the cell, a less damage from enzyamatic toxicity, and proliferating as same as in vivo, may be obtained with high efficiency by treating a sample derived from biological tissue with the composition of the present invention, which provides an effective dispersion of the cell. In particular, a cell having a high proliferation rate may be obtained with high efficiency from hard tissue such as scirrhous cancer tissue by using the composition of the present invention. Various analyses, such as assessment of drugs such as anticancer agent; and analysis of biological material such as functional macromolecule including gene, protein, and sugar chain, may be carried out by culturing the cell obtained by using the compositions of the present invention sterically or in a three-dimensional culture method forming agglomerates because the cell proliferates as same as in vivo. In addition, various analyzes may be carried out from a smaller amount of cell by culturing the cell obtained by using the composition of the present invention with embedding into a droplet gel.

### BRIEF DESCRIPTION OF DRAWINGS

FIG. 1 is a photograph comparing the efficiency of digestion of pseudo-interstitial tissue.

FIG. 2 is a graph comparing the toxicity of the composition to HCT-116 cell and PC-14 cell.

FIG. 3 is a photograph comparing the proliferation of cell which is prepared by using the composition containing an enzyme.

FIG. 4 is a graph comparing the cell proliferation after treatment with the composition containing an enzyme.

FIG. 5-1 is a graph comparing the susceptibility of cell to various drugs after treatment with the composition containing an enzyme.

FIG. 5-2 is a graph comparing the susceptibility of cell to various drugs after treatment with the composition containing an enzyme.

FIG. 6 is a graph showing T/C (%) values of the composition of Example 1 and the composition of Comparative Example 1 on one-on-one plot.

FIG. 7 is a photograph comparing the result of neutral red staining carried out after culturing the cells obtained by treating the composition containing an enzyme in the droplet gel.

### MODE FOR CARRYING OUT THE INVENTION

The present invention provides a composition for dispersing biological tissue. A collagenase activity of the composition of the present invention in a formulation concentration is 0.30 U/mL to 10 U/mL, preferably, 0.30 U/mL to 5 U/mL, more preferably 0.30 U/mg to 1 U/mg, as determined by a method for determining FALGPA-degrading activity. A method of determining FALGPA-degrading activity is shown in the following Test Example 2 as a method of determining FALGPA-degrading activity. A value determined in this protocol (U/mL) is considered as a collagenase activity. FALGPA is N-(3-[2-Furyl]acryloyl)-Leu-Gly-Pro-Ala. A trypsin activity of the composition of the present invention in a formulation concentration is 0 U/mL to 30 U/mL, preferably 0 U/mL to 20 U/mL, more preferably 0 U/mL to 10 U/mL, as determined by a method for determining BAEE hydrolytic activity. A method of determining BAEE hydrolytic activity is shown in the following Test Example 2 as a method of determining BAEE hydrolytic activity. A value determined in this protocol (U/mL) is considered as a trypsin activity. BAEE is N-a-Benzoyl-L-arginine ethyl ester hydrochloride. A formulation concentration is a concentration of the composition of the present invention in dispersing biological tissue by using the composition of the present invention.

The composition of the present invention may be prepared by mixing commercially available enzymes such as coliagenase, dispase, and hyaluronidase, then determining a collagenase activity and a trypsin activity of the mixture by the method of determining FALGPA-degrading activity and the method of determining BASE hydrolytic activity, and then adjusting the FALGPA-degrading activity and BASE hydrolytic activity to desired range. Any collagenase, such as Clostridium origin and actinomycete origin, may be used. In addition, the collagenase having any purity may be used, and it is preferable that a crude collagenase is contained. Furthermore, the composition of the present invention may contain various degrading enzymes such as hyaluronidase, deoxyribonuclease, elastase, dispase, and thermolysin. Preferably, the composition contains dispase. Since dispase degrades type IV collagen, and fibronectin, which are cell scaffold in biological body, cell is more efficiently obtained. Moreover, the composition of the present invention may contain a trypsin inhibitor in order to control trypsin activity. Examples of the trypsin inhibitor include serum. Cytotoxicity in the composition of the present invention may be reduced by using serum.

The composition of the present invention may be used for obtaining a cell by dispersing a treated sample derived from biological tissue. Examples of the biological body include human, and non-human mammal such as mouse, rat, guinea pig, hamster, rabbit, dog, cat, sheep, pig, goat, cattle, and monkey. Examples of the biological tissue include cancer tissue and normal tissue. Examples of cancer include gastrointestinal cancer, head and neck cancer, breast cancer, lung cancer, cancerous pleurisy and peritonitis, cervical cancer, endometrial cancer, and ovarian cancer. The composition of the present invention is particularly suitable for digestion and dispersion of scirrhous cancer. Examples of the sample derived from biological tissue include all or a part of surgical material and all or a part of biopsy sample. As the surgical material, for example, a tissue to be excised in a surgical resection for the purpose of treatment may be used. In addition, a tissue collected in a minimally invasive sampling method as a test excision or a test centesis may be used for the purpose of pathological diagnosis, treatment of disease, and determination of prognosis. Examples of the tissue collected in a minimally invasive sampling method include samples obtained from various biopsy, thoracoscopic or laparoscopic material, ascite, and pleural effusion. The sample may be subjected to a mechanical separation process such as cutting with scissor, tweezer or razor after collection from biological body. In addition, the sample may be washed with wash solution containing a medium component or antibiotic. Furthermore, the sample may be a paste obtained by mince treatment after collection from a cancer patient.

Dispersion of the sample derived from biological tissue may be carried out by mixing the composition of the present invention and the sample from biological tissue and treating at 25 to 40° for 3 minutes to 72 hours. More preferably, the dispersion of the sample derived from biological tissue may be carried out by treating for 5 minutes to 24 hours. An amount of the sample derived from biological tissue at the mixing is, for example, 0.1 to 5 g/10 mL. A collagenase activity at the mixing is 0.30 U/ml, to 10 U/mL, preferably, 0.30 U/mL to 5 U/mL, more preferably 0.30 U/mL to 1 U/mL, as determined by a method for measuring FALGPA-degrading activity. A method determining FALGPA-degrading activity is as mentioned above. A trypsin activity at the mixing is 0 U/mL to 30 U/mL, preferably 0 U/mL, to 20 U/mL, more preferably 0 U/mL to 10 U/mL, as determined by a method for determining BADE hydrolytic activity. A method of determining FALGPA-degrading activity is as mentioned above.

After the dispersion, a cell may be obtained from the mixture of the composition of the present invention and the sample derived from biological tissue. The dispersed mixture containing the sample derived from biological tissue is preferably treated with serum in order to reduce a cytotoxicity of the enzyme contained in the composition of the present invention and improve a proliferation property of the obtained cell. It may also be treated with a metal chelating agent such as EDTA in order to reduce an action of the enzyme. In addition, the enzyme solution may be removed by centrifugation in order to remove the enzyme. A filtration may be carried out for recovering the cell by using a filter such as nylon mesh and cell strainer. Furthermore, a solution in which a sample derived from biological tissue is dispersed may be seeded onto a culture medium and cultured to selectively harvest the proliferated cell. Culture may be carried out on a support. Cell adhesion factors may be applied in layers to the support onto which a sample derived from biological tissue. Examples of the cell adhesion factors include an extracellular matrix such as various types of collagen, fibronectin, laminin, vitronectin, cadherin, gelatin, peptide, and integrin. These may be used alone or in combination of two or more. More preferably, various types of collagen may be used because the cell adhesion and cell stretching are improved. It is particularly preferable to use type I collagen or type IV collagen among the various types of collagen. Cell adhesion factors to be applied the surface of the support may be the same as the gelling agent in the droplet gel. Harvest of the cell adhered to the support may be carried out by, for example, removing the culture medium containing blood cells and unwanted cellular components, and then adding cell exfoliating agent to remove the cell adhered to the support. Examples of the cell exfoliating agent include EDTA-trypsin. An exfoliation of the cell adhered to the support may be carried out by adding an exfoliating agent for an applied matter if the applied matter is on the support. The exfoliating agent is, for example, a collagenase if the applied matter on the support is a collagen. The exfoliation of cell by addition of collagenase provides less damage to living cell because the collagen gel layer itself will be enzymatically degraded before the enzyme acts to the living cell.

A result similar to in vivo may be evaluated by culturing in two-dimensional culture method, in sterically grown method or in three-dimensional culture method forming the agglomerates, and then evaluating the culture result. A result more similar to in vivo may be obtained by culturing in three-dimensional culture method, and then evaluating the culture result. Examples of the three-dimensional culture method include, but not limited to, a method of embedding into an extracellular matrix such as collagen or Matrigel, a method of culturing in an incubator having a low adhesive culturing surface, a method of culturing in an incubator having a U-bottom culturing surface, a method of culturing in an incubator having a micropatterned culturing surface, and a method of culturing in a culture droplet. Various analyzes may be carried out from a smaller amount of cell by culturing the cell obtained by using the composition of the present invention with embedding into the droplet gel. Examples of the droplet gel include a gel having a shape of convex surface on a planar substrate. Examples of a volume of the droplet gel include 3 to 300 μL, 3 to 150 μL, 5 to 100 μL, and 15 to 50 μL. A height of the droplet gel is, for example, 2 mm or less. Examples of the droplet gel include a gel showing transmittance percent ranging from 1 to 95% of transparency for 400 nm light. Examples of a viscosity of the droplet gel include 50 to 2000 centipoise and 100 to 1000 centipoise, from the viewpoint of compatibility and ease of handling, maintenance of shape of the droplet gel. The droplet gel may contain a gelling agent. Examples of the gelling agent include collagen such as acid-soluble type I collagen; extracellular matrix such as Matrigel; and soft agar. An amount of collagen in the droplet gel is, for example, 0.1 to 2.0% by weight, from the viewpoint of maintaining a shape of the droplet gel. Furthermore, the droplet gel may contain polymeric material such as polysaccharide and other extracellular matrix, and medium components such as serum. A pH of the droplet gel may be adjusted to, for example, pH 6.2 to 7.6, or pH 6.8 to 7.4, with a buffer. A salt strength or ionic strength of the droplet gel is, for example, 100 to 180 mmol, or 140 to 160 mmol.

The droplet gel may be used with embedding the cell obtained by treating a sample derived from biological tissue with the composition of the present invention. A concentration of the cell embedded in the droplet gel is, for example, 10² to 10⁷ cells/mL, preferably 10³ to 10⁶ cells/mL. The droplet gel embedding the cell may be prepared by, for example, mixing components such as cell and a solution containing a gelling agent, then cooling the obtained mixed liquid with ice, then putting a drop of the mixed liquid on a substrate, and then standing it at 30 to 45° C. for 30 minutes to 2 hours. The substrate has a surface which may fix the droplet gel. Examples of the substrate include culture dish such as Petri dish and multi-dish; conventional culture vessel such as flask; culture plate such as cover slip or cell disk which is a thin plate made of glass or plastic. The substrate is, preferably optically transparent on the point that an evaluation of cell culture result is easy. The culture with embedding in droplet gel is carried out, for example, 1 to 10 days, preferably 3 to 8 days.

Examples of culture result to be evaluated include a change in the number of viable cells before and after the culture, and a change in cell product before and after the culture. Examples of the cell product include nucleic acid such as DNA and RNA, and protein. A drug may be added to the droplet gel in cell culture. In this case, an effect of the drug on the cell may be evaluated by comparing cells before and after the culture, or by comparing cells cultured with and without an addition of the drug.

Examples of the drug evaluation include a method of evaluating an effect of the drug on the cell, which includes contacting a solution containing the drug to the droplet gel embedding the cell, then contacting the droplet gel embedding the cell to a medium, then culturing the cell in the droplet gel embedding the cell, and then evaluating the result of the culture.

Examples of drugs in the drug evaluation include therapeutic agent, prophylactic agent, and improving agent. Examples of diseases include cancer. Examples of therapeutic agent, prophylactic agent, and improving agent for cancer include anticancer agent which acts directly on cancer cell, and agent which acts indirectly on cancer cell, but shows an effect such as inhibition of a proliferation of cancer cell, decrease of action of cancer cell, and killing cancer cell by working together with an immune cell in the biological body or other drugs. Examples of the anticancer agent include, antimetabolite such as 5-FU; irinotecan-based anticancer agent such as SN-38; microtubule depolymerization inhibitor such as docetaxel; platinum formulation such as cisplatin and 1-oxaliplatin. Other examples include molecularly targeted drug which selectively modifies growth factor and their receptor involved in cell proliferation, and molecule and enzyme involved in cell proliferation, cell cycle, apoptosis and signaling thereof to obtain anticancer effect. For example, trastuzumab, cetuximab, and gefitinib, which act on a growth factor receptor, imatinib, and crizotinib, which act on a signal transduction of fusion gene, and bevacizumab, which inhibit angiogenesis of cancer tissue, may be exemplified. Examples of the other drugs include prodrug of anti-cancer agent, agent which modulates an intracellular metabolic enzyme activity involved in the metabolism of the anti-cancer agent or prodrug thereof, and immunotherapeutic agent.

Examples of the action to be evaluated in the drug evaluation include an action involved in a possibility of obtaining treatment, prevention or improvement in the biological body when the drug is administered to the biological body derived from the cell. Examples of the effect of the treatment, prevention, or improvement include reduction of diseased cell proliferation, cell damage, and reduction of tissue size.

In the process of the drug evaluation, contact of a droplet gel and a solution containing a drug is preferably carried out by overlaying the solution containing the drug onto the droplet gel with covering the entire of the droplet gel with the solution containing the drug so that the droplet gel is not dried to become a flat dried product. The solution containing the drug to be contacted may contain a medium such as serum medium other than the drug. Concentration of the drug in the solution is, preferably a drug concentration in the vicinity of cell when the drug is administered to a biological body which is origin of the cell.

Cell culture after contacting with the solution containing the drug as carried out by contacting the medium with the droplet gel embedding the cell. The medium to be contacted is preferably a liquid medium. The liquid medium to be contacted is, preferably serum-free medium from the point to suppress a proliferation of fibroblast, or to maintain and express a function of fibroblast. A contact with the liquid medium is carried out, preferably by covering the entire of the droplet gel with the liquid medium so that the droplet gel is not dried to become a flat dried product. Culture period is, for example, 1 to 10 days, preferably 3 to 8 days. The droplet gel to be contacted with the liquid medium may be obtained by washing to remove the drug after contacting with the solution containing the drug.

The drug evaluation may include contacting the medium and the droplet gel embedding the cell, then culturing, and then evaluating the culture result. This evaluation of the culture result may be carried out, for example, by comparing the number of viable cells before and after cultivation, or by comparing the number of viable cells after cultivation with and without adding a drug. A measurement of the number of viable cells may be carried out by visual observation using a microscope. Alternatively, a measurement of the number of viable cells may be carried out by subjecting viable cells to staining for selectively staining viable cells, and by measuring the color development by staining. Examples of the staining method for selectively staining viable cells include method of using cellular phagocytosis such as neutral red staining, a method of using intracellular enzymatic activity such as latex particle staining method and fluorescein diacetate staining method, and staining method using other fluorescent agent. The cell after the staining may be fixed, such as by formalin fixation. This enables to carry out a highly sensitive staining by temporarily preventing an elution of dye. The droplet gel after the staining may be dried. This enables to prevent deterioration and degradation. The drying of the droplet gel may be carried out, for example, by air drying, or by forced drying by heating at about 10 to 50° C. A measurement of a color development by staining may be carried out by taking a picture, and then digitizing the picture to evaluate. An evaluation after digitizing may include a correction of the numerical value based on a shape of the image of the stained cells. A cancer cell tends to provide a dark and mass form image, and a fibroblast tends to provide a pale and thin fibrous image. A viable cancer cell may be detected more accurately and easily by carrying out a correction to select a number of mass form staining image.

Another method for the evaluation of the culture result is carried out, for example, by comparing a variation of cell gene expression before and after the culture, or presence and absence of contact with the drug. The variation of gene expression may be determined by analyzing mRNA expression in the cultured cell with known methods such as real time RT-PCR method and method using DNA chip. As the gene of interest, all genes may be compared, or a gene involved in a target molecule of the drug or the function thereof, a gene involved in the drug metabolism, or a gene involved in the cell cycle, or survival or death of the cell may be compared individually or in combination. A drug required for the analysis of gene may be added into the droplet gel during culture.

Still another method is carried out, for example, by comparing proteins, such as cell surface antigen, receptor protein, and drug-metabolizing enzyme, expressed by the cell before and after the culture, or presence and absence of contact with the drug. Well known methods such as immunostaining method, ELISA method and enzymatic activity measuring method may be used for detecting protein. Pathology specimens prepared by fixing and embedding the recovered droplet gel after the culture may be compared with an immunohistochemical staining method. A drug required for the analysis of protein may be added into the droplet gel during culture.

Yet another method is carried out, for example, by comparing the mutant genes in the cell before and after the culture, or presence and absence of contact with the drug. Well known methods such as well known genetic analysis including PCR and DNA sequencing, and in situ hybridization. A drug required for the analysis of mutant gene may be added into the droplet gel during culture.

A method for obtaining a cell by using a composition of the present invention and a method of evaluating a result of cell culture may be carried out by using a kit including the composition of the present invention. The kit may Include a collagen solution and a liquid medium in addition to the composition of the present invention. The collagen solution which may be included in the kit is used for preparing a droplet gel by mixing with a cell obtained from a sample derived from biological tissue. Examples of collagen to be included in the collagen solution include acid soluble type I collagen and type IV collagen, and pepsin-soluble type I collagen and type III collagen. The liquid medium which may be included in the kit is used for culturing cell in the droplet gel. The liquid medium may be a concentrated medium. Examples of the concentrated medium include a base medium for mammalian cell culture such as McCoy's 5A, RPMI-1640, D-MEM, MEM, MCDB-131, Ham's 1-12, D-MEM/1-12 and Medium-199.

In addition, the kit may include a reconstruction buffer. The reconstitution buffer neutralizes an acid-soluble collagen solution to solidify the droplet gel. Examples of the reconstitution buffer include sodium hydroxide aqueous solution adjusted to pH 7 to 10. Furthermore, the kit may include a support for seeding and culturing a sample derived from biological tissue. Examples of the support include collagen gel flask and tube for culture support. Examples of the tube for culture support include a flat-bottomed tube having a shape as obtained by cutting a portion of a tube container so as to have a gentle angle to the central axis of the container and so as to form a flat cutting face having a surface area of 0.01 to 25.0 cm², where the flat cutting face is used as a supporting base, and where the surface of the supporting base is a portion of sticking and culturing a cell. The kit may include a medium for culturing on the support in addition to the liquid medium for culturing the cell in the droplet gel. Examples of the medium for culturing on the support include a culture medium having a proliferating action and physiological activity-retaining action on an animal cell derived from biological tissue as well as a killing action and/or multiplication-inhibition action on bacterium. More specifically, the examples include a medium obtained by adding 5 to 20% of fetal bovine serum (FBS), and if necessary, various growth factors, to Ham's F-12 or D-MEM, or D-MEM/F-12 mixture. These media may contain an antibiotic.

In addition, the kit may include a cytological staining agent for evaluating the cell culture result. Examples of the cytological staining agent include a staining agent utilizing a phagocytosis of cell, such as neutral red. More favorable is the Neutral Red which utilizes phagocytosis to lysosome and has high correlation to cell life. The kit may also include other components in addition to the above mentioned components.

### EXAMPLES

Hereinafter, the present invention will be more specifically illustrated by the following examples. However, the present invention is not limited thereto.

### Test Example 1

### Anticancer Agent Susceptibility Test

In the following test examples, the anticancer agent susceptibility test was carried out based on the following procedure unless otherwise specified.

### 1. Sample Washing:

Sample is recovered from a tissue of solid cancer in a patient, such as gastrointestinal cancer including eastric cancer, bowel cancer, pancreatic cancer; breast cancer; lung cancer; head and neck cancer; cancerous pleurisy and peritonitis; cervical cancer; endometrial cancer and ovarian cancer. Bacteria which adhere to the sample surface are removed by the following method. First, each 20 mL of medium solution containing antibiotic (sample washing solution) is put into three 10 cm dishes. The surface of the sample is sufficiently washed in the sample washing solution in the first dish. The sample is sequentially washed in the second dish and then the third dish. The sample washing solution used is prepared by adding pentcillin (manufactured by Toyama Chemical Co., Ltd., for pentcillin injection) at a final concentration of 1 mg/mL with respect to base medium, kanamycin (manufactured by Meiji Seika Kaisha, Ltd., kanamycin sulfate injection) at a final concentration of 0.5 mg/mL with respect to the base medium, and Anpoterishin B (manufactured by Wako Pure Chemical Industries, Ltd.) at a final concentration of 2.5 μg/mL to DF culture medium (DF: mixed culture medium of 1 volume of Dulbecco's Modified Eagle (DME) broth and 1 volume of Ham's F12 culture medium).

### 2. Fine-Cutting Treatment of Tissue:

The washed sample is put into new dish and quickly fine-cut with scissors and forceps to make tumor tissue about 3 to 5 mm cube on the dish.

### 3. Mince Treatment of Tissue:

The fine-cut sample is minced on the dish with razor blades sandwiching the needle holder to make the fine-cut sample to paste. To the minced tumor tissue, 20 mL DF culture medium is added, and the tissue and the DF culture medium are recovered to 50 mL centrifuge tube. Another 10 mL DF culture medium is added to recover the tumor tissue adhered to the dish. It is subjected to a centrifugation for 3 minutes at 400×g with a tabletop centrifuge.

### 4. Tissue Dispersion:

After the centrifugation, the supernatant is removed by aspiration. To the centrifuged sediment, 9 mL DF culture medium is added, and the centrifuge tube is shaken to loosen the piece of tissue. Depending on the enzyme composition, 10% FBS (fetal bovine serum) may be added. The cell dispersion solution with a formulation concentration is prepared by adding 1 mL enzyme composition for cell dispersion adjusted to 10-fold concentration of the formulation concentration. The solution is subjected to stirring and shaking for about 1 to 2 hours in 37° C. incubator.

### 5. Recovery:

To the solution, 10 mL DR culture medium is added to make 20 ml total volume. The solution is centrifuged for 3 minutes at 400×g, and then the supernatant is removed. After removing the supernatant, the centrifuge tube is lightly shaken to loosen the cell mass, then 10 mL culture medium is added thereto, and then the cell mass is further loosen by repeating strong aspiration and blowing-out with a pipette. The suspension containing the cell is filtered by using a nylon mesh having a pore size of 300 μm. The centrifuge tube and nylon mesh are rinsed out by 10 mL DE culture medium.

### 6. Preculture:

The cell obtained in the above recovery treatment is recovered by centrifugation, and the supernatant is removed by aspiration. The cell pellet after the centrifugation is suspended to 5 mL preculture medium (PCM-1) of Primaster kit (manufactured by KURABO INDUSTRIES LTD.). The PCM-1 culture medium in which the cells are suspended is seeded to collagen gel flask. Culture is carried out by standing the flask in a CO₂ incubator, overnight. After the overnight culture, the culture medium containing blood cells and unwanted cellular components is removed by aspiration. Engraftment of tumor cells to the collagen gel flask is observed.

### 7. Cell Recovery:

The culture medium in the collagen gel flask is removed by aspiration, and the cell is washed with 5 mL DF culture medium, and then 2 mL DF culture medium is added. An enzyme solution with a formulation concentration is prepared by adding 0.2 mL enzyme composition for cell dispersion adjusted to 10-fold concentration of the formulation concentration. The collagen gel in the flask is dissolved by shaking at 37° C. for 15 to 30 minutes. Exfoliated cells from the collagen gel flask are collected into 50 mL centrifuge tube. If cell adhesion to the flask is observed, the flask is shaken for 5 minutes after adding 3 mL EDTA-trypsin. After confirming the release of the cells, the flask is rinsed out by adding 5 mL of 10% serum medium, and then the cells are collected into 50 mL centrifuge tube. After adding 10 mL DC culture medium, the cells are recovered by centrifugation.

### 8. Embedding:

The supernatant is removed, and the sediment after the centrifugation is subjected to a treatment with 2 mL EDTA-trypsin solution for 3 to 7 minutes, then 10 mL of 10% serum medium is added and the mixture is treated by repeating aspiration and blowing-out with a pipette, and then the cell suspension liquid is filtered with a nylon mesh having a pore size of 100 μm. The centrifuge tube and nylon mesh are rinsed out enough by adding 10 mL DC culture medium. After centrifuging the filtrate, the supernatant was removed by aspiration to recover the cells. A collagen solution is added to the recovered cells and mixed. The collagen solution containing the recovered cells is cooled with ice. The cells mixed collagen solution is cooled with ice, and three drops per 1 well are put on a plate with a micropipette adjusted to 30 μL/drop. The collagen drop is made to gel by standing in a CO₂ incubator at 37° C. for 1 hour. After the gelation, DF medium containing 10% FBS is overlaid at 3 mL/well. A serum-free medium (PCM-2) may be used. After the overlaying, culture is carried out in a CO₂ incubator, overnight.

### 9. Drug Contact:

After the overnight culture, concentrated drug solution is added and mixed to the medium so that the drug concentration becomes a predetermined concentration. A contact culture for predetermined time depending on the drug is carried out in a CO₂ incubator.

### 10. Drug Removal and Culture:

The culture medium is removed by aspiration after completion of the drug contact, and serum-free medium (PCM-2) of Primaster kit (manufactured by KURABO INDUSTRIES LTD.) is overlaid 4 mL to each well to carry out serum-free culture for 5 days.

### 11. Evaluation:

After the serum-free culture, 40 μL neutral red (NE) solution is added into each well. Incubation is carried out in a CO₂ incubator for 2 hours to stain the cell. After the neutral red staining, the culture medium containing the stain solution is removed by aspiration. After the removal of the culture medium, 4 mL of 10% neutral formalin solution is added and cell is fixed at room temperature for about 1 hour. After the cell fixation, the neutral formalin solution is removed. The culture plate is immersed into tap water for 20 minutes to wash. After the washing with water, water on the plate is drained and the plate is air-dried. Image analytical processing of the cell fixed with neutral red is carried out. A method for image analytical processing disclosed in JP H10-115612 A (ASSAY OF CANCER CELL) is used for the image analytical processing.

### Test Example 2

The compositions of Example 1 and Comparative Example 1 having trypsin activity and collagenase activity as shown in table 1 were prepared by mixing commercially available collagenase, dispase, hyaluronidase, and deoxyribonuclease. The prepared compositions were used for tissue dispersion as mentioned in Test Example 1, step 4.

TABLE 1

| | Collagenase activity | Trypsin activity |
|---|---|---|
| | (U/mL) | (U/mL) |
| Example 1 | 0.367 | 9.8 |
| Comparative | 0.242 | 38.4 |
| Example 1 | | |

Collagenase activity of the compositions of Example 1 and Comparative Example 1 was determined by the following method for determining FALGPA-degrading activity.

### 1. Method for Determining FALGPA-Degrading Activity:

The following method is shown on the Sigma-Aldrich's website (http://www.sigmaaldrich.com/technical-documents/protocols/biology/enzymatic-assay-of-collagenase-using-n-3-2furylacryloyl-leu-gly-pro-ala.html).

### (1) Abbreviations:

TABLE 2

| | |
|---|---|
| FALGPA | N-(3-[2-Furyl]acryloyl)-Leu-Gly-Pro-Ala |
| FAL | N-(3-[2-Furyl]acryloyl)-Leu |
| Leu | Leucine |
| Gly | Glycine |
| Pro | Proline |
| Ala | Alanine |

### (2) Principle:

An activity is calculated by measuring the decrease variate of absorbance at 345 nm (A345 nm) derived from FALGPA when FALGPA is degraded into FAL and Gly-Pro-Ala by the action of the collagenase.

### (3) Method:

a. Reagents:

(a) Reagent B 50 mM Tricine, 10 mM CaCl₂, 400 mM NaCl, ph 7.5 (25° C.) buffer:

It is prepared by dissolving 0.896 g tricine (Sigma-Aldrich, T0377), 2.34 g NaCl (Sigma-Aldrich, S9888), and 0.147 g CaCl₂.2H₂O (Sigma-Aldrich, C3881) to 80 mL distilled water, adjusting the solution to pH 7.5 (25° C.) by adding 1M NaOH solution (Sigma-Aldrich, S2567), or 1M HCl solution (Sigma-Aldrich, H3162), and then adjusting the total volume of the solution to 100 mL with distilled water.

(b) Reagent C 1.0 mM N-(3-[2-furyl]acryloyl)-Leu-Gly-Pro-Ala (FALGPA):

It is prepared by adding 9.6 mg FALGPA (Sigma-Aldrich, F5135) to 20 mL solution of reagent A, and completely dissolving with stirring for 30 minutes or more.

(c) Reagent D distilled water:

(d) Reagent E enzyme solution:

It is prepared by dissolving the enzyme into distilled water so as to be 5 to 10 times the concentration in use.

b. Conditions:

Reaction solution pH=7.5, reaction temperature=25° C., absorbance=A345 nm, optical path length=1 cm

c. Reagent Composition of the Reaction Solution and Operation:

TABLE 3

| Reagent | Blank test | Main test |
|---|---|---|
| B | 2.9 mL | 2.9 mL |
| C | 0.1 mL | — |
| C | — | 0.1 mL |

To a cell having 1 cm optical path length, 2.9 mL reagent B is put, and warmed to 25° C. Once A345 nm is stable, 0.1 mL reagent C (blank test) or reagent D (main test) is added and immediately mixed, and decrease of A345 nm is recorded for 5 minutes at 25° C.

(4) Definition and calculation method of active unit

An amount of enzyme which hydrolyzes 1.0 μmole FALGPA for 1 minute under the above-mentioned condition, at 25° C. and pH 7.5, and in the presence of calcium ion, is defined as 1 FALGPA unit. FALGPA unit is calculated by the following equation.

FALGPA units/mL={(E1−E2)×3/(F×0.1)}/0.53

Symbols or values in the above formula show the following.

TABLE 4

| | |
|---|---|
| E1: | Change of absorbance per minute in main test |
| E2: | Change of absorbance per minute in blank test |
| 0.53: | Molecular extinction coefficient of FALGPA (Δ_{E}345/cm/mM) |
| 3: | Total amount of reaction solution (mL) |
| 0.1: | Amount of enzyme solution (mL) |
| F: | Ratio of enzyme solution to concentration in use |

Trypsin activity of the compositions of Example 1 and Comparative Example 1 was determined by the following method of determining BAEE hydrolytic activity.

### 2. Method for Determining BAEE Hydrolytic Activity:

The following method is shown in Sigma-Aldrich's website (http://www.sigmaaldrich.com/technical-documents/protocols/biology/enzymatic-assay-of-trypsin.html).

(1) Abbreviation: BAEE=Nα-benzoyl-L-arginine ethyl hydrochloride

### (2) Principle:

An activity is calculated by measuring the increase variate of absorbance at 253 nm (A253 nm) when the BAEE is hydrolyzed to Nα-Benzoyl-L-arginine and ethanol by the action of trypsin.

### (3) Method:

a. Reagents:

(A) Reagent A 67 mM sodium phosphate buffer, pH 7.5 (25° C.)

It is prepared by dissolving 0.804 g Sodium dihydrogen phosphate (Sigma-Aldrich, S0751) into 80 mL distilled water, adjusting the solution to pH 7.6 (25° C.) by adding 1M NaOH solution (Sigma-Aldrich, S2567), and then adjusting the total volume of the solution to 100 mL with distilled water.

(B) Reagent B 0.25 mM Ne-benzoyl-L-arginine ethyl hydrochloride:

It is prepared by adding and dissolving 4.3 g Nα-benzoyl-L-arginine ethyl hydrochloride (Sigma-Aldrich, B4500) into 50 mL solution of reagent A.

(d) reagent C distilled water:

(d) reagent D enzyme solution:

It is prepared by dissolving the enzyme into distilled water so as to be 5 to 10 times the concentration in use.

b. Conditions:

Reaction solution pH=7.6, reaction temperature=25° C., absorbance=A253 nm, optical path length=1 cm

c. Reagent Composition of the Reaction Solution and Operation:

TABLE 5

| Reagent | Blank test | Main test |
|---|---|---|
| B | 3.0 mL | 3.0 mL |
| C | 0.2 mL | — |
| D | — | 0.2 mL |

To a cell having 1 cm optical path length, 3.0 mL reagent B is put, and warmed to 25° C. Once A253 nm is stable, 0.1 mL reagent C (blank test) or reagent D (main test) is added and immediately mixed, and decrease of A253 nm is recorded for 5 minutes at 25° C. d. Definition and calculation method of active unit

An amount of enzyme which increases A253 nm by 0.001 for 1 minute under the above-mentioned condition is defined as 1 BAEE unit. BAEE unit is calculated by the following equation.

BAEE units/mL=(E1−E2)/{0.001×(F×0.1)}

Symbols or values in the above formula show the following.

TABLE 6

| | |
|---|---|
| E1: | Change of absorbance per minute in main test |
| E2: | Change of absorbance per minute in blank test |
| 0.001: | Increment in A253 nm by 1 unit enzyme per minute under |
| | the condition at pH 7.6, 25° C., 3.2 mL reaction solution |
| | and 1 cm light path. |
| 0.1: | Amount of enzyme solution (mL) |
| F: | Ratio of enzyme solution to concentration in use |

### Test Example 3

### Comparison of Enzyme Digestion Activities

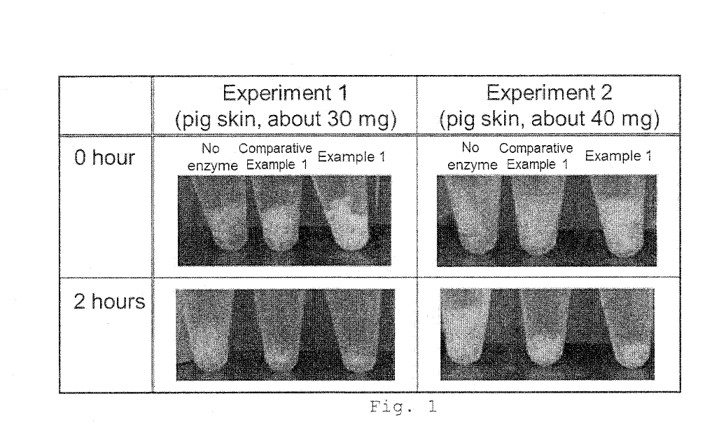

Pigskin was used to assess a digestibility. The efficiency of pigskin digestion of the composition of Example 1 and that of the composition of Comparative Example 1 were compared. More specifically, 0.1 mL of the composition of Example 1 or Comparative Example 1 and 0.9 mL DF medium containing 10% FBS were mixed and added to cut pigskin, and shaken at 37° C., and then the size of the pigskin was observed. The status after 0 hour and 2 hours are shown in FIG. 1.

As shown in FIG. 1, better digestion result was obtained in the composition of Example 1 than the composition of Comparative Example 1.

### Test Example 4

Cancer tissues from 10 gastric cancer samples and 10 bowel cancer samples were digested by using the composition of Example 1 or the composition of Comparative Example 1. Comparative evaluation was carried out by visually observing an amount of the undecomposed residue. The result is shown in the following Table 7. In the following Table 7, the column “Example 1” shows the number of samples that the amount of the undecomposed residue in Example 1 was less than the amount of the undecomposed residue in Comparative Example 1, the column “Comparative Example 1” shows the number of samples that the amount of the undecomposed residue in Comparative Example 1 was less than the amount of the undecomposed residue in Example 1. The column “Similar extent” shows the number of samples that no significant difference was observed in the amount of the undecomposed residue between both Examples.

TABLE 7

| | Gastric cancer | Bowel cancer |
|---|---|---|
| Number of samples | 10 | 10 |
| Example 1 | 5 | 5 |
| Comparative Example 1 | 0 | 0 |
| Similar extent | 5 | 5 |

As shown in Table 7, the composition of Example 1, compared with the composition of Comparative Example 1, showed better digestibility against gastric cancer tissue and bowel cancer tissue. Thus, the composition of Example 1 showed higher digestibility regardless of the type of cancer tissues.

### Test Example 5

### Comparison of Cytotoxicity Against Cell Lines

The cytotoxicity with the composition of Example 1 and that of Comparative Example 1 was compared by using HCT-116 cell from colon cancer and PC-14 cell from lung cancer. More specifically, suspension (DE medium containing 10% FBS) containing approximately 500,000 cells/mL HOT-116 cells or PC-14 cells was mixed to the composition of Example 1 or that of Comparative Example 1, and the mixture was incubated at 37° C., and the number of cells was confirmed every two hours.

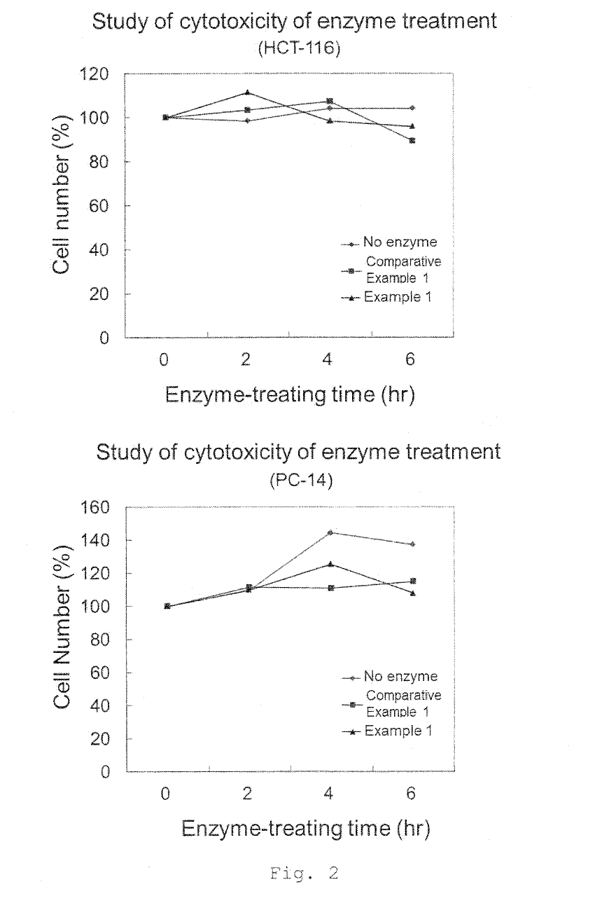

The result is shown in FIG. 2, FIG. 2 is a graph having vertical axis which shows the number of cells % with assuming that the number of cells at 0 hour is 100%. As shown in FIG. 2, for the HOT-116 cell, both the composition of Example 1 and the composition of Comparative Example 1 showed little increase or decrease in the number of cells similar to no enzyme. For the PC-14 cell, the number of cells was increased in no enzyme, and the number of cells was slightly increased and not decreased than the initial number of cells in both the composition of Example 1 and the composition of Comparative Example 1. Thus, no significant difference was observed in the cytotoxicity between the composition of Example 1 and the composition of Comparative Example 1. The composition of Example 1 showed low cytotoxicity similar to the composition of Comparative Example 1.

### Test Example 6

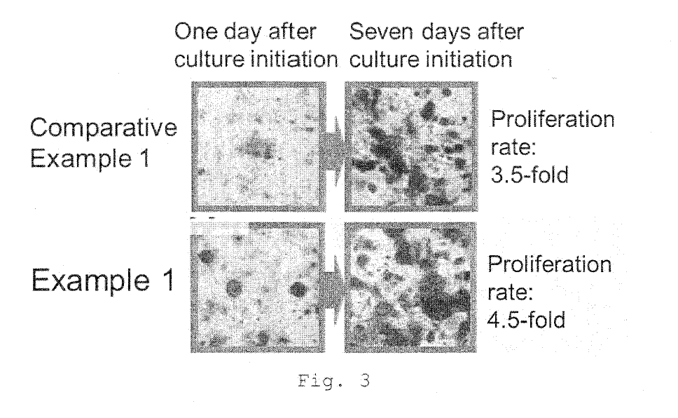

Cancer cells were recovered from cancer tissues according to steps 2 to 7 of Test Example 1 by using the composition of Example 1 and the composition of Comparative Example 1. The obtained cancer cells were cultured with embedded in collagen gel drop for 7 days according to steps 8 and 10 of Test. Example 1. Images of stained by neutral red (NR) of the cells at one day and 7 days after culture initiation are shown in FIG. 3.

As shown in FIG. 3, the growth rate at 7 days after culture initiation was 4.5-fold in the case of using the composition of Example 1 while the growth rate was 3.5-fold in the case of using the composition of Comparative Example 1. Thus, the composition of Example 1 provided cells suitable for culture in collagen drops than the composition of Comparative Example 1.

### Test Example 7

Tissues of bowel cancer, gastric cancer, and lung cancer were made to paste form according to steps 2 and 3 of Test Example 1, then the obtained tissues were divided into two groups, then cancer cells were recovered by using the composition of Example 1 or the composition of Comparative Example 1 according to steps 4 and 5 of Test Example 1, then the cells was precultured overnight according to step 6 of Test Example 1, then cells were recovered according to step 7 of Test Example 1, and then the number of viable cells was measured by trypan blue staining method. The measured number of cells is shown in the following Tables 8-1 to 8-3. In Tables 8-1 to 8-3, “tissue weight” shows the weight per one group of cancer tissue which was used for recovering the cell. “Comparison of recovered cell count” shows a value obtained by dividing the number of cells in Example 1 by the number of cells in Comparative Example 1 from the same sample. The number of cells which have not been damaged is more accurately measured by measuring the number of cells immediately after the preculture, rather than the number of cells in the recovered paste-form tissue pieces.

TABLE 8-1
Gastric cancer

| | | Recovered viable cell | | Comparison of |
|---|---|---|---|---|
| | Tissue | count (×10⁵ cell) | | recovered cell count |
| | weight per | Comparative | | Example 1/ |
| No. | group | Example 1 | Example 1 | Comparative Example 1 |
| 1 | 0.39 | 2.3 | 2.9 | 1.26 |
| 2 | 0.45 | 2.2 | 4.0 | 1.82 |
| 3 | 0.48 | 5.7 | 6.7 | 1.18 |
| 4 | 0.43 | 6.5 | 8.7 | 1.34 |
| 5 | 0.48 | 0.8 | 1.2 | 1.50 |
| 6 | 0.38 | 27.3 | 40.2 | 1.47 |
| 7 | 0.29 | 7.3 | 7.3 | 1.00 |
| 8 | 0.14 | 2.4 | 2.8 | 1.17 |
| 9 | 0.51 | 1.3 | 4.4 | 3.38 |
| 10 | 0.42 | 3.5 | 7.0 | 2.00 |

TABLE 8-2
Bowel cancer

| | | Recovered viable cell | | Comparison of |
|---|---|---|---|---|
| | Tissue | count (×10⁵ cell) | | recovered cell count |
| | weight per | Comparative | | Example 1/ |
| No. | group | Example 1 | Example 1 | Comparative Example 1 |
| 1 | 0.29 | 1.9 | 2.6 | 1.37 |
| 2 | 0.40 | 3.4 | 3.3 | 0.97 |
| 3 | 0.45 | 3.5 | 3.2 | 0.91 |
| 4 | 0.27 | 1.1 | 1.1 | 1.00 |
| 5 | 0.44 | 0.7 | 1.6 | 2.29 |
| 6 | 0.31 | 1.6 | 1.9 | 1.19 |
| 7 | 0.17 | 0.5 | 1.0 | 2.00 |
| 8 | 0.49 | 2.3 | 2.8 | 1.22 |
| 9 | 0.23 | 4.9 | 6.6 | 1.35 |
| 10 | 0.34 | 1.8 | 2.6 | 1.44 |

TABLE 8-3
Lung cancer

| | | Recovered viable cell | | Comparison of |
|---|---|---|---|---|
| | Tissue | count (×10⁵ cell) | | recovered cell count |
| | weight per | Comparative | | Example 1/ |
| No. | group | Example 1 | Example 1 | Comparative Example 1 |
| 1 | 0.28 | 0.4 | 1.0 | 2.50 |
| 2 | 0.17 | 0.6 | 1.1 | 1.83 |
| 3 | 0.77 | 28.5 | 37.2 | 1.31 |
| 4 | 0.34 | 7.2 | 8.4 | 1.17 |
| 5 | 0.24 | 6.2 | 5.8 | 0.94 |
| 6 | 0.22 | 3.2 | 3.4 | 1.06 |

As shown in Table 8, the composition of Example 1 enabled to recover same or larger number of cells as compared to the composition of Comparative Example 5 when assuming that both are considered as similar when the relative difference in the number of recovered cells is less than 25%, and that the one is considered as larger than the other when it is 25% or more.

### Test Example 8

Cancer cells were recovered from gastric cancer tissues of 10 subjects, bowel cancer tissues of 10 subjects, lung cancer tissues of 6 subjects, breast cancer tissues of 2 subjects, and pancreatic cancer tissues of 2 subjects according to steps 1 to 5 in Test Example 1 by using the composition of Example 1 and the composition of Comparative Example 1. The recovered cancer cells were precultured according to step 6 of Test Example 1 by using the collagen gel flask (manufactured by Kurabo Industries Ltd.). After the preculture, the cells were recovered from the collagen gel flask according to step 7 of Test Example 1. The number of the recovered cells was measured and the number of cells in the case of Example 1 and that in the case of Comparative Example 1 were compared. The result is shown in the following Table 9. In Table 9, the column “Example 1” shows the number of samples that the number of the recovered cells in the case of Example 1 was 25% or more larger than that of Comparative Example 1, the column “Comparative Example 1” shows the number of samples that the number of the recovered cells in the case of Comparative Example 1 was 25% or more larger than that of Example 1. The column “Similar extent” shows less than 25% relative difference in the number of the recovered cells between the case of Example 1 and that of Comparative Example 1.

TABLE 9

| | Gastric | Bowel | Lung | Breast | pancreas |
|---|---|---|---|---|---|
| | cancer | cancer | cancer | cancer | cancer |
| Number of samples | 10 | 10 | 6 | 6 | 6 |
| Example 1 | 7 | 5 | 3 | 3 | 4 |
| Comparative | 0 | 0 | 0 | 0 | 0 |
| Example 1 | | | | | |
| Similar extent | 3 | 5 | 3 | 3 | 2 |

As shown in Table 9, the composition of Example 1 enabled to recover same or larger number of cells as compared with the composition of Comparative Example 1 for all 4 types of cancer cells. In addition, the number of cells which were adhered and remained in the collagen gel flask in the recovery from the flask in the case of using the composition of Example 1 was smaller than in the case of using the composition of Comparative Example 1. Thus, the composition of Example 1 enabled to achieve efficient recovery. In the case of the recovery of the cancer cells from breast cancer tissue, the amount of undecomposed residue after the enzyme reaction in the case of using the composition of Comparative example 1 was smaller than in the case of using the composition of Example 1. However, the amount of the recovered cells from the collagen gel flask in the case of using the composition of Example 1 was larger. This may suggest that the composition of Example enables to expose cancer cells from a breast cancer tissue without a completely digestion of the breast tissue, and the composition of Example 1 enables to recover cancer cells from a breast cancer tissue.

Thus, the composition of Example 1 enabled to recover the cancer cells more efficiently regardless of the type of cancer than the composition of Comparative Example 1.

### Test Example 9

An effect of the composition of Example 1 and the composition of Comparative Example 1 to a cell proliferation of cultured cell line was confirmed by using HOT-116 derived from colon cancer and PC-14 derived from lung cancer as the cultured cell lines. More specifically, the cell lines were incubated in an enzyme solution containing the composition of Comparative Example 1 or Example 1 for 2 hours, then the collagen drop embedding culture was carried out according to steps 8 and 10 of Test Example 1, and then the cell proliferation after 24, 48, and 120 hours was confirmed.

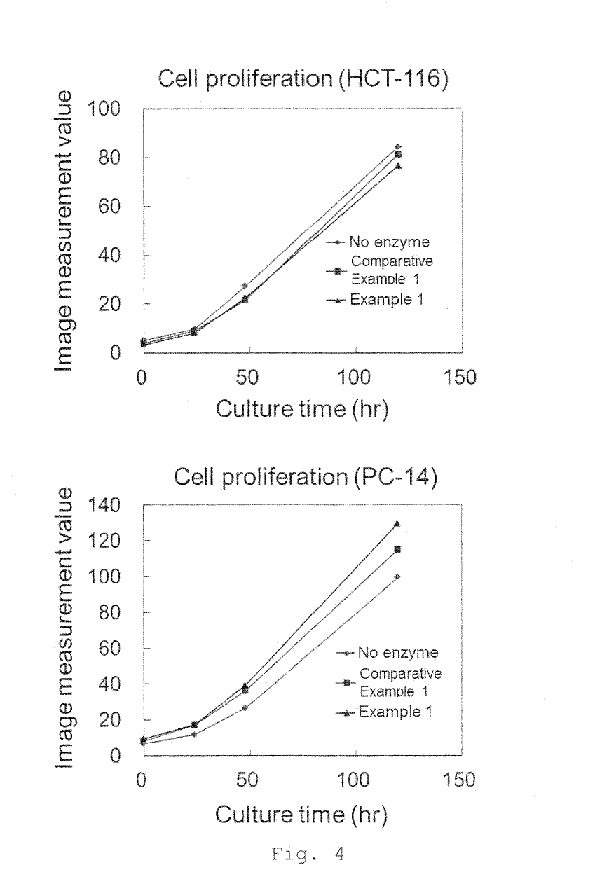

The result is shown in FIG. 4. As shown in FIG. 4, no difference in cell proliferation was observed in the cases of no enzyme, Example 1 and Comparative Example 1. Thus, the composition of Example 1 enabled to obtain a cell having good proliferation in the droplet gel.

### Test Example 10

### Anticancer Agent Susceptibility Test Using Cultured Cell

The composition of Example 1 or Comparative Example 1 was contacted to HOT-116 cell derived from lung cancer and PC-14 cell derived from colon cancer with assuming a tissue digestion, then the anticancer agent susceptibility test (CD-DST method) was carried out according to the method of Test Example 1 and values of image analysis were determined to compare various drug susceptibilities.

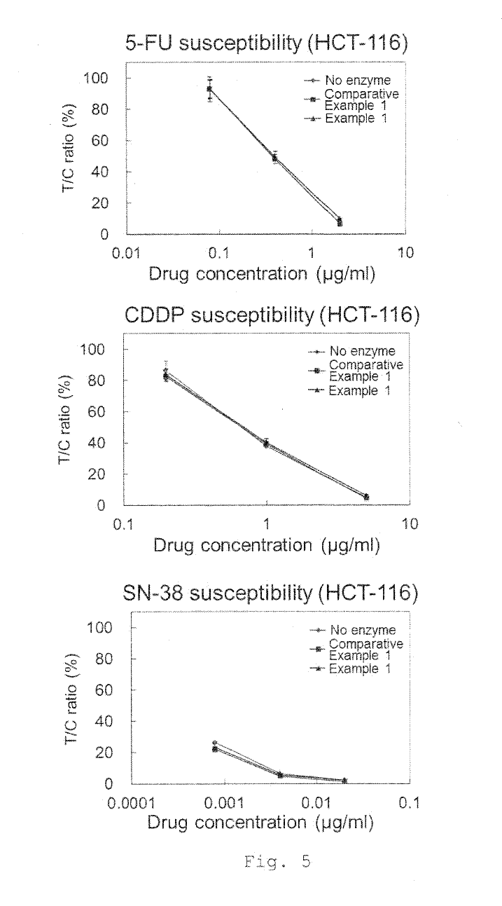

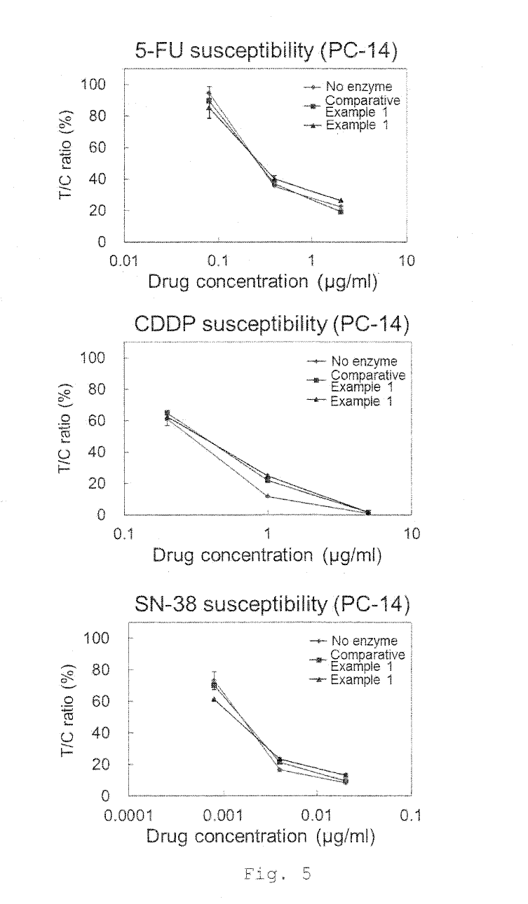

The result is shown in FIG. 5. T/C ratio (%) in FIG. 5 is a value calculated by dividing a value of image analysis after 120 hours at each drug concentration by a value of image analysis without adding a drug. The experimental result in the case of using the cell without an enzyme solution treatment was shown as untreated. As shown in FIG. 5, in the case of using the composition of Example 1, both HCT-116 cell and PC-14 cell showed drug susceptibility to 5-EU, cisplatin (CDDP), and SN-38 at the same level as in the case of using the composition of Comparative Example 1. When the drug susceptibility to docetaxel and oxaliplatin was determined, the drug susceptibilities were similar in the cases of the composition of Example 1, the composition of Comparative Example 1 and the untreated. Thus, the composition of Example 1 enabled to obtain a cell showing similar drug susceptibility to various anticancer agents such as 5-FU, CDDP, SN-38, docetaxel, and oxaliplatin.

### Test Example 11

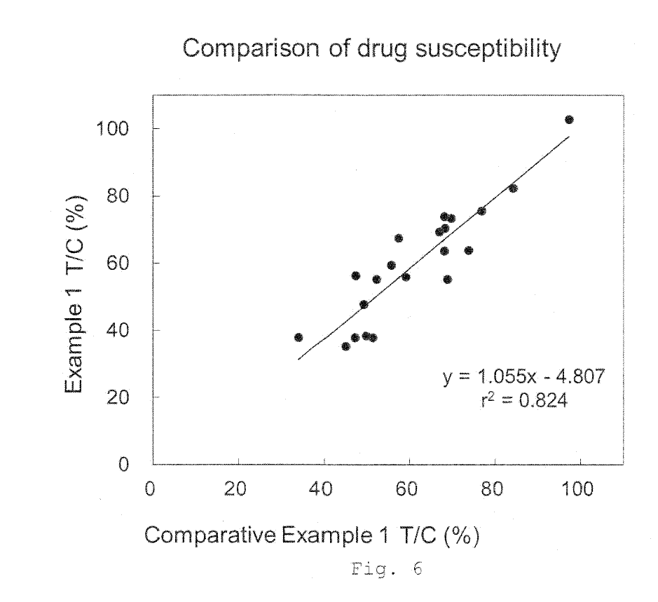

T/C (%) values were obtained according to the method of Test Example 10 by using the composition of Example 1 or the composition of Comparative Example 1 for bowel cancer, and the obtained values were plotted to one-on-one plot. The result is shown in FIG. 6.

As shown in FIG. 6, the regression line showing high correlation with slope 1 was obtained from the plot data. From this fact, it can be said that an equivalent drug susceptibility evaluation is carried out in the composition of Example 1 and the composition of Comparative Example 1 in the case of bowel cancer.

### Test Example 12

### Success Rate of Anti-Cancer Drug Susceptibility Test (CD-DST Method)

The anti-cancer drug susceptibility test (CD-DST method) was carried out according to the method of Test Example 1 by using the composition of Example it and the composition of Comparative Example 1. CD-DST method was respectively carried out for the plurality of subjects.

As a result, CD-DST method was not completed in some subjects. The reason why the method was not completed was that cancer cell proliferation with forming colony was not observed, and valid numeric data by the image analysis was not obtained.. The number of subjects in which the method was completed without such a problem was considered as the number of success. The ratio of the number of success to the number of implementation was considered as success rate (%). The numerical result such as success rate is shown in the following Table 10.

TABLE 10

| | Gastric cancer | | Bowel cancer | |
|---|---|---|---|---|
| Number of | Comparative | | Comparative | |
| samples | Example 1 | Example 1 | Example 1 | Example 1 |
| Succeeded/ | 5/10 | 8/10 | 9/10 | 9/10 |
| Implemented | | | | |
| Success rate | 50 | 80 | 90 | 90 |
| (%) | | | | |

As shown in Table 10, high success rate of CD-DST method was obtained for any type of cancer by using the composition of Example 1. Thus, it is found that the composition of Example 1 is beneficial in order to recover the cancer cell suitable for susceptibility test.

### Test Example 13

### Comparison of Collagen-Gel-Drop Culture Cell Staining of Gastric Cancer Cases

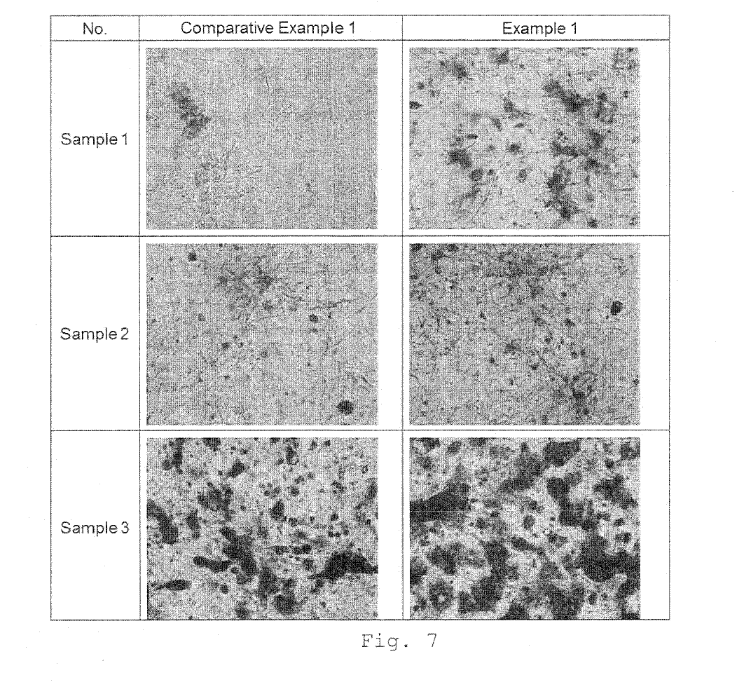

Among cancer cells of which proliferation was observed after the culture of gastric cancer in Test Example 12, cancer cells from three subjects, respectively, were stained with neutral red. The staining images are shown in FIG. 7.

As shown in FIG. 7, Example 1 showed better proliferation of cancer cell, densely stained, in sample 1. However, valid value data from image analysis was not obtained and CD-DST method was not completed in Comparative Example 1 was also considered as one factor that larger amount of cells was recovered in the case of Example 1.

Example 1 showed better proliferation of cancer cell, densely stained, after 144 hours culture in samples 2 and 3 although similar number of cells was seeded. In sample 2, valid value date was obtained and CD-DST method was completed in Example 1 while valid value data from image analysis was not obtained and CD-DST method was not completed in Comparative Example 1.

As described above, a cell which proliferated in a manner suitable for anticancer drug susceptibility test was obtained from a tissue of various cancers, such as gastric cancer, bowel cancer, and breast cancer in the case of using the composition of Example 1.

1. A composition for dispersing biological tissue, wherein a collagenase activity of the composition in a formulation solution is 0.30 U/mL to 10 U/mL as determined by a method for measuring FALGPA-degrading activity, and wherein a trypsin activity of the composition in the formulation solution is 0 U/mL to 30 U/mL as determined by a method for measuring BAEE hydrolytic activity.

2. The composition according to claim 1, for a drug assessment.

3. The composition according to claim 1, wherein the biological tissue is cancer tissue.

4. A method for obtaining a cell derived from biological tissue, comprising treating a sample derived from the biological tissue with the composition according to claim 1.

5. A method for evaluating a cell culture result, wherein the cell is treated with the composition according to claim 1.

6. The method according to claim 5, wherein the cell culture result is a result from two-dimensional culture.

7. The method according to claim 5, wherein the cell culture result is a result from three-dimensional culture.

8. The method according to claim 7, wherein the three-dimensional culture is carried out in a droplet gel.

9. A kit for carrying out the method according to claim 4, comprising said composition for dispensing biological tissue.
